(19) United States
(12) Patent Application Publication
(10) Pub. No.: US 2025/0319299 A1
(43) Pub. Date: Oct. 16, 2025

(54) CARDIAC SUPPORT SYSTEM FLOW MEASUREMENT USING PRESSURE SENSORS

(71) Applicant: KARDION GMBH, Stuttgart (DE)

(72) Inventors: Ingo Stotz, Ditzingen (DE); Thomas Alexander Schlebusch, Renningen (DE)

(21) Appl. No.: 19/253,571

(22) Filed: Jun. 27, 2025

Related U.S. Application Data
(63) Continuation of application No. 15/734,003, filed on Jun. 3, 2021, now Pat. No. 12,377,256.

(30) Foreign Application Priority Data
Jun. 6, 2018 (DE) 102018208862.4

(51) Int. Cl. A61M 60/148; A61M 60/178; A61M 60/216; A61M 60/546; A61M 60/554; A61M 60/816

(52) U.S. Cl. CPC A61M 60/148; A61M 60/178; A61M 60/216; A61M 60/546; A61M 60/554; A61M 60/816; A61M 2205/0244; A61M 2205/3351

(57) ABSTRACT

A cardiac support system may include a tubular structure configured to permit fluid to flow therethrough. The cardiac support system may include a first pressure sensor connected to the tubular structure and configured to determine at least one of a static pressure or a total pressure in a first region of the cardiac support system having a first cross-sectional area. The cardiac support system may include a second pressure sensor connected to the tubular structure and configured to determine at least one of a static pressure or a total pressure in a second region of the cardiac support system having a second cross-sectional area of a different size relative to the first cross-sectional area.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application is a continuation of U.S. patent application Ser. No. 15/734,003, filed Jun. 3, 2021, titled CARDIAC SUPPORT SYSTEM FLOW MEASUREMENT USING PRESSURE SENSORS, which is a 371 National Phase Entry of PCT Application No. PCT/EP2019/064784, filed Jun. 6, 2019, titled IMPLANTABLE VASCULAR SUPPORT SYSTEM, which claims priority to German Patent Application No. 10 2018 208 862.4, filed Jun. 6, 2018, titled IMPLANTABLE VASCULAR SUPPORT SYSTEM, the entire contents of which are incorporated by reference herein for all purposes and forms a part of this specification.

### BACKGROUND

### Field

The invention relates to an implantable vascular support system, a method for determining at least a flow velocity or a fluid volume flow of a fluid flowing through an implanted vascular support system, and a use of two pressure sensors of an implantable vascular support system. The invention can in particular be used in (fully) implanted left ventricular assist devices (LVAD).

### Description of the Related Art

Implanted left ventricular assist devices (LVAD) exist primarily in two design variants. The first are (percutaneous) minimally-invasive left ventricular assist devices. The second variant are left ventricular assist devices which are invasively implanted under an opening in the rib cage. The first variant conveys blood directly from the left ventricle into the aorta, because the (percutaneous) minimally invasive left ventricular assist device is positioned centrally in the aortic valve. The second variant conveys the blood from the left ventricle into the aorta via a bypass tube.

The task of a cardiac support system is to convey blood. The so-called cardiac output (CO, usually expressed in liters per minute) is of high clinical relevance here. Simply put, the cardiac output refers to the total volume flow of blood (out of a ventricle), in particular from the left ventricle to the aorta. The initial objective is therefore to obtain this parameter as a measured value during the operation of a cardiac support system.

Depending on the level of support, which describes the proportion of volume flow conveyed by a conveying means, such as a pump of the support system, to the total volume flow of blood from the ventricle to the aorta, a specific amount of volume flow reaches the aorta via the physiological path through the aortic valve. The cardiac output or the total volume flow (Q_{CO}) from the ventricle to the aorta is therefore usually the sum of the pump volume flow (Qₚ) and the aortic valve volume flow (Qₐ).

In the clinical setting, the use of dilution methods is an established procedure for determining the cardiac output (Q_{CO}). However, these dilutions methods all rely on a transcutaneously inserted catheter and can therefore only provide cardiac output measurement data during cardiac surgery. Whereas the determination of the cardiac output by a support system is difficult to implement, the pump volume flow (Qₚ) can be determined by means of suitable components of the support system. For high levels of support, the aortic valve volume flow (Qₐ) approaches zero or becomes negligibly small, so that Qₚ approximately equals CO or the pump volume flow (Qₚ) can be used as an approximation for the cardiac output (Q_{CO}). Correlating the operating parameters of the support system, particularly the electrical power consumption, possibly supplemented by other physiological parameters, such as the blood pressure, is an established procedure for measuring the pump volume flow (Qₚ).

Since these methods are based on statistical assumptions and the underlying pump characteristic map of the support system used, the correlated Qₚ can be error-prone. Increasing the measurement quality of the parameter Qₚ is therefore desirable.

### SUMMARY

Based on this, the underlying object of the invention is to optimize an implantable vascular support system, in particular also with regard to the arrangement and the use of sensors.

Proposed here is an implantable vascular support system comprising:
a fluid channel, which passes through the support system and through which fluid can flow,
a first pressure sensor, which is disposed and configured to determine at least a static pressure or a total pressure in the region of the support system,
a second pressure sensor, which is disposed and configured to determine at least a static pressure or a total pressure in the region of the fluid channel.

The solution proposed here advantageously enables the calculation of the pump volume flow with the aid of pressure sensors integrated in the support system, in particular in the inlet cannula of the support system. The particularly advantageous aspect of the implementation using pressure sensors, in comparison with ultrasonic sensor systems, for example, is the low price, the small space requirement and the simple evaluation method.

The vascular support system is preferably a cardiac support system, particularly preferably a ventricular support system. The support system is routinely used to support the conveyance of blood in the circulatory system of humans, e.g. a patient. The support system can be disposed at least partially in a blood vessel. The blood vessel is the aorta, for example, in particular in the case of a left ventricular assist device, or the common trunk (truncus pulmonalis) into the two pulmonary arteries, in particular in the case of a right ventricular assist device. The support system is preferably disposed at the outlet of the left ventricle of the heart or the left ventricle. The support system is particularly preferably disposed in aortic valve position.

The support system is preferably a left ventricular cardiac support system (LVAD) or a percutaneous, minimally invasive left ventricular assist device. The support system is particularly preferably configured and/or suited to being disposed at least partially in a ventricle, preferably in the left ventricle of a heart, and/or in an aorta, in particular in aortic valve position.

The support system is furthermore preferably fully implantable. In other words, this means in particular that the means required for determination, in particular the pressure sensors, are located entirely inside the body of the patient and remain there. The support system can also have a multipart design, i.e. comprise a plurality of components that can be disposed spaced apart from one another, so that the pressure sensors and a processing unit (measuring unit), for example, can be disposed separated from one another by a wire. In the multipart design, the processing unit disposed separate from the pressure sensors can likewise be implanted or disposed outside the patient's body. Either way, it is not absolutely necessary for a processing unit to also be disposed in the body of the patient. For example, the support system can be implanted such that a processing unit (the support system) is disposed on the patient's skin or outside the patient's body and a connection to the pressure sensors disposed in the body is established. Fully implanted in this context means in particular that the means required for determination (here the pressure sensors) are located entirely inside the patient's body and remain there. This advantageously makes it possible to determine the pump volume flow even outside of cardiac surgery and/or estimate the cardiac output even outside of cardiac surgery.

The support system further preferably comprises a tube (or a cannula), in particular an inlet tube or inlet cannula, a flow machine, such as a pump, and/or an electric motor. The electric motor is a routine component of the flow machine. The (inlet) tube or the (inlet) cannula is preferably configured such that, in the implanted state, it can guide fluid from a (left) ventricle of a heart to the flow machine. The support system is preferably elongated and/or hose-like. The tube (or the cannula) and the flow machine are preferably provided in the region of oppositely disposed ends of the support system. The tube preferably forms or surrounds the fluid channel.

According to one advantageous configuration, it is proposed that a first pressure sensor be disposed in the region of an outer side of the support system. The first pressure sensor is preferably disposed in or on an outer side of an inlet cannula of the support system, which forms or surrounds the fluid channel.

According to one advantageous configuration, it is proposed that a first pressure sensor be disposed in or on a channel interior surface of the fluid channel. The fluid channel is preferably formed or surrounded by an inlet cannula of the support system.

According to one advantageous configuration, it is proposed that a second pressure sensor be disposed in or on a channel interior surface of the fluid channel. The first pressure sensor and the second pressure sensor are preferably disposed spaced apart from one another in or on a channel interior surface of the fluid channel.

According to one advantageous configuration, it is proposed that a first pressure sensor be disposed in the region of a first channel cross-section through which fluid can flow and the second pressure sensor be disposed in the region of a second channel cross-section through which fluid can flow different from the first channel cross-section through which fluid can flow. A first pressure sensor is preferably disposed in or on a channel interior surface of the fluid channel in the region of a or in a first (known) channel cross-section through which fluid can flow and a second pressure sensor is disposed in or on the channel interior surface of the fluid channel in the region of a or in a second (known) channel cross-section through which fluid can flow different from the first channel cross-section through which fluid can flow.

According to another advantageous configuration, it is proposed that at least the first pressure sensor or the second pressure sensor be implemented as a MEMS pressure sensor. MEMS stands for microelectromechanical system.

According to a further aspect, a method for determining at least a flow velocity or a fluid volume flow of a fluid flowing through an implanted vascular support system is proposed, comprising the following steps:
a) determining at least a static pressure or a total pressure in the region of the support system by means of a first pressure sensor,
b) determining at least a static pressure or a total pressure in the region of a fluid channel, which passes through the support system and through which fluid can flow, by means of a second pressure sensor,
c) determining at least the flow velocity or the fluid volume flow using the pressures determined in Steps a) and b).

In other words, the fluid volume flow relates in particular to a fluid volume flow which flows (only) through the support system itself, e.g. through an (inlet) tube or an (inlet) cannula of the support system. The fluid volume flow is furthermore preferably the volume flow of the fluid flowing through the fluid channel. The flow velocity is therefore in particular the flow velocity of the fluid flowing through the fluid channel.

This fluid volume flow is usually the so-called pump volume flow (Qₚ), which quantifies only the flow through the support system itself. If this value is known in addition to the total volume flow or cardiac output (Q_{CO}), the so-called level of support can be calculated from the ratio of Qₚ to Q_{CO} (i.e., Qₚ/Q_{CO}). To determine the fluid volume flow, the obtained flow velocity can be multiplied, for example, with a cross-section of the support system through which fluid can flow, in particular a tube or cannula cross-section through which fluid can flow.

In Step c), the fluid volume flow can be determined based on Bernoulli's pressure equation for incompressible fluids, for example. The equation is:

$${p}_{t} = p + \frac{ρ}{2} \cdot {v}^{2} = const .$$

$$\approx const .$$

In the above equation, pₜ is the total pressure, p is the static pressure, ρ is the fluid density and v is the flow velocity. The equation therefore states that the total pressure pₜ consists of a static component and a kinematic component. For a flow with small, i.e. negligible, friction losses, this total pressure is constant and a velocity difference of the flow can therefore be calculated by measuring the pressure at two positions.

If the cross-sectional area A through which the fluid flows is furthermore known at each of these positions, the volume flow can be determined with the known fluid density ρ using the continuity equation for incompressible fluids. The corresponding equation for the fluid volume flow Q is:

$$Q = \frac{{A}_{2} \cdot \sqrt{\frac{2 \cdot Δ p}{ρ}}}{\sqrt{1 - {( \frac{{A}_{2}}{{A}_{1}} )}^{2}}}$$

In Step c), the flow velocity can likewise be determined based on Bernoulli's pressure equation for incompressible fluids, for example. In this context, it is particularly advantageous if the first pressure sensor determines a static pressure and the second pressure sensor determines a total pressure. Since the pressure sensors are both integrated in or on the support system, it can be assumed that the static pressure measured by means of the first pressure sensor is also representative of the static pressure at the second pressure sensor. The flow velocity v can then be determined to:

$$v = \sqrt{\frac{2}{ρ} \cdot ( {p}_{t} - p )}$$

For this purpose, the static pressure p is advantageously measured with the first pressure sensor and the total pressure pₜ is measured with the second pressure sensor.

According to one advantageous configuration, it is proposed that a static pressure be determined in Step a). A static pressure is preferably determined in both Step a) and in Step b).

According to one advantageous configuration, it is proposed that a total pressure in the region of the fluid channel be determined in Step b). Preferably, a static pressure is determined in Step a) and a total pressure is determined in Step b). In this case, the flow velocity v can be determined comparatively easily in Step c) according to the above equation.

Preferably, in Step a), at least a static pressure or a total pressure is determined in the region of a or in a first (known) channel cross-section of the support system through which fluid can flow, in particular the fluid channel, by means of the first pressure sensor. Further preferably, in Step b), at least a static pressure or a total pressure is determined in the region of a or in a second (known) channel cross-section of the support system through which fluid can flow, in particular the fluid channel, by means of the first pressure sensor.

According to one advantageous configuration, it is proposed that a flow cross-section of the fluid be changed between the steps a) and b). There is preferably a flow cross-sectional widening or a flow cross-sectional narrowing between the first pressure sensor and the second pressure sensor.

According to a further aspect, the use of two pressure sensors of an implantable vascular support system to determine at least a flow velocity or a fluid volume flow of a fluid flowing through the support system is proposed.

### BRIEF DESCRIPTION OF THE DRAWINGS

The details, features and advantageous configurations discussed in connection with the support system can correspondingly also occur in the method and/or the use presented here and vice versa. In this respect, reference is made in full to the statements there for a more detailed characterization of the features.

The solution presented here as well as its technical environment are explained in more detail below with reference to the figures. It is important to note that the invention is not intended to be limited by the design examples shown. In particular, unless explicitly stated otherwise, it is also possible to extract partial aspects of the facts explained in the figures and to combine them with other components and/or insights from other figures and/or the present description. The figures show schematically:

FIG. 1a: a percutaneous, minimally invasive left ventricular assist device,

FIG. 1b: a left ventricular assist device invasively implanted under an opening in the rib cage,

FIG. 2: an implanted vascular support system,

FIG. 3: the support system according to FIG. 2 in a detail view,

FIG. 4: an illustration of a fluid channel through which fluid can flow, and

FIG. 5: a sequence of a method presented here in a routine operating sequence.

### DETAILED DESCRIPTION

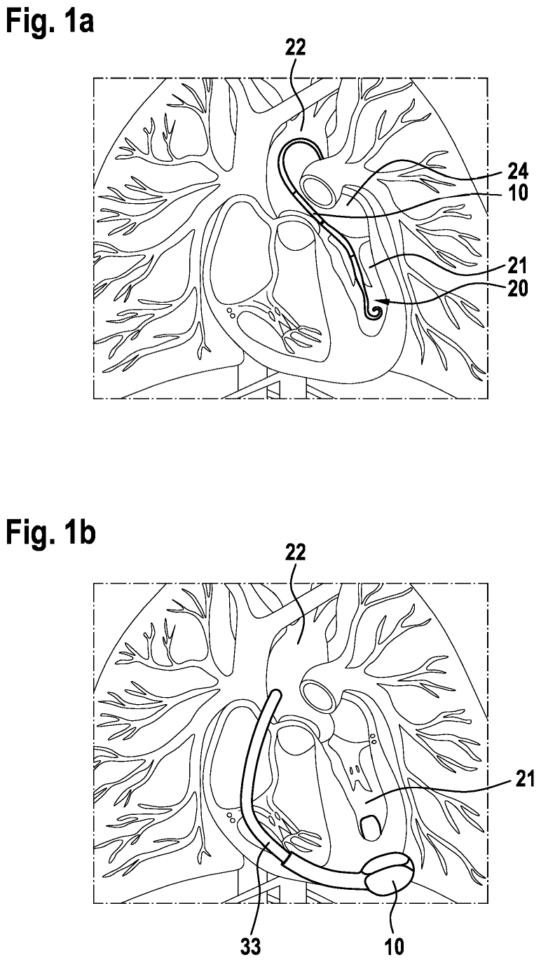

The vascular support system is preferably a ventricular and/or cardiac support system or a cardiac support system. Two particularly advantageous forms of cardiac support systems are systems which are placed in the aorta, such as the one depicted in FIG. 1a, and systems which are placed apically, such as the one depicted in FIG. 1b.

The support system 10 is described in the following using a left ventricular assist device (LVAD) as an example. Implanted left ventricular assist devices (LVAD) exist primarily in two design variants, as shown in FIGS. 1b and 1b. FIG. 1a shows a (percutaneous) minimally invasive left ventricular assist device 10, whereas FIG. 1b shows a left ventricular assist device 10 invasively implanted under an opening in the rib cage. The variant of FIG. 1a conveys blood directly from the left ventricle 21 through the atrium 24 into the aorta 22, because the (percutaneous) minimally invasive left ventricular assist device 10 is positioned centrally in the aortic valve. The variant of FIG. 1b conveys the blood from the left ventricle 21 into the aorta 22 via a bypass tube 33.

Depending on the level of support, which describes the proportion of volume flow conveyed by a conveying means, such as a pump of the support system, to the total volume flow of blood from the ventricle 21 to the aorta 22, a specific amount of volume flow reaches the aorta 22 via the physiological path through the aortic valve. The cardiac output or the total volume flow (Q_{CO}) from the ventricle 21 to the aorta 22 is therefore usually the sum of the pump volume flow (Qₚ) and the aortic valve volume flow (Qₐ).

$${Q}_{CO} = {Q}_{p} + {Q}_{a}$$

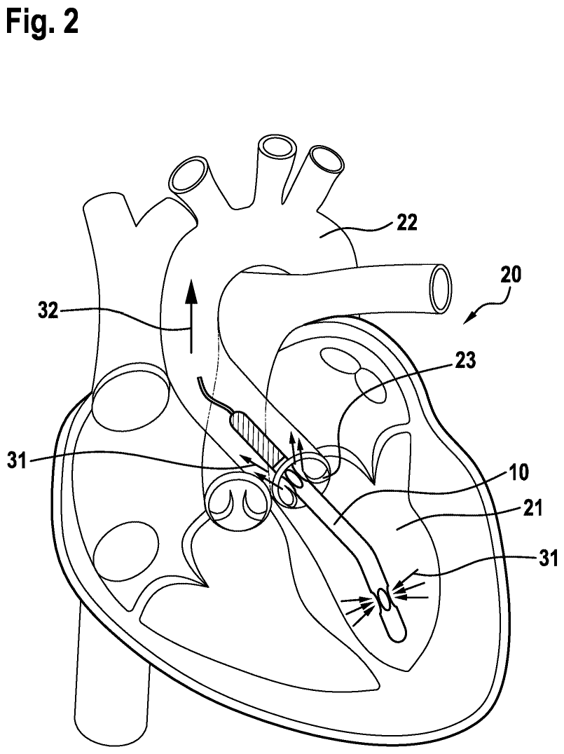

FIG. 2 schematically shows an implanted vascular support system 10. The cardiac support system 10 is implanted in a heart 20. The reference signs are used consistently, so that reference can be made in full to the above statements.

FIG. 2 shows a heart 20 with a minimally invasive cardiac support system (VAD pump) 10 as an example. The VAD is positioned centrally in the aortic valves 23 between the ventricle 21 and the aorta 22 and conveys a blood volume flow 31 from the ventricle 21 into the aorta 22 to support the cardiac output 32 of the patient.

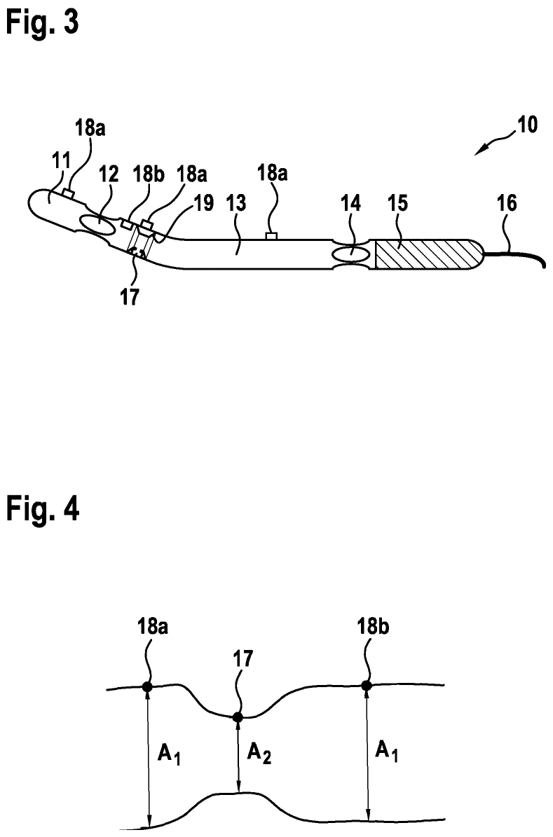

FIG. 3 schematically shows the support system 10 according to FIG. 2 in a detail view. The reference signs are used consistently, so that reference can be made in full to the above statements.

FIG. 3 schematically shows an implantable vascular support system 10 comprising:
a fluid channel 13, which passes through the support system 10 and through which fluid can flow,
a first pressure sensor 18a or 18b, which is disposed and configured to determine at least a static pressure or a total pressure in the region of the support system 10,
a second pressure sensor 17, which is disposed and configured to determine at least a static pressure or a total pressure in the region of the fluid channel 13.

According to the illustration of FIG. 3, as an example, the support system 10 further comprises a tip 11, which can contain sensors (for example temperature, pressure), an inlet cage with openings 12 for drawing in a liquid (here: blood), an inlet cannula 13 for delivering the blood to a (not shown) pump element in an impeller cage 14 provided with an opening, from which the blood can again exit the inlet cannula 13. Connected to this, as an example, is a drive (electric motor) 15 and an electrical supply cable 16.

In order to be able to estimate the cardiac output, the blood volume flow 31 through the inlet cannula 13 of the support system 10, which is also referred to as the so-called pump volume flow (symbol Qₚ), is to be measured here. For this purpose, it is proposed here that two pressure sensors 17 and 18a/18b be integrated in or on the support system 10.

In Configuration A, the first pressure sensor 18a is positioned on the outside of the support system or the VAD pump 10, preferably in a region with a negligible flow velocity, e.g. on the outside of the tip 11, on the outside of a constriction 19 or on the outside of the inlet cannula 13. In other words, this means in particular that, in Configuration A, the first pressure sensor 18a is disposed in the region of an outer side of the support system 10.

In Configuration B, the positioning of the first pressure sensor 18b differs as shown in FIG. 4. Here, the first pressure sensor 18b is seated inside the inlet cannula 13 at a position with a known flow cross-section A₁. In other words, this means in particular that, in Configuration B, the first pressure sensor 18b is disposed in or on a channel interior surface of the fluid channel 13.

In both Configurations A and B, another (second) pressure sensor 17 is used, which is disposed in or on a channel interior surface of the fluid channel (13). In FIG. 3, as an example, the second pressure sensor 17 is positioned in the inlet cannula 13 and preferably in an annular constriction 19 with the known flow cross-section A₂.

FIG. 3 according to Configuration B therefore also shows that a first pressure sensor 18b is disposed in or on a channel interior surface of the fluid channel 13 in the region of a first channel cross-section Ai through which fluid can flow and a second pressure sensor 17 is disposed in or on the channel interior surface of the fluid channel 13 in the region of a second channel cross-section A₂ through which fluid can flow different from the first channel cross-section through which fluid can flow.

The pressure measured by the pressure sensors 17 and 18a,b now depends on the flow velocity prevailing there. For a known fluid with a known density ρ, for a frictionless flow or a flow that has negligible losses between the pressure sensors, it follows that:
for Configuration A with the known cross-sectional area A₂ at the position of the second pressure sensor 17:

$$Q = {A}_{2} \cdot \sqrt{\frac{2 \cdot Δ p}{ρ}}$$

for Configuration B with the known cross-sectional areas A₁ at the position of the first pressure sensor 18a,b and A₂ at the position of the second pressure sensor 17:

$$Q = \frac{{A}_{2} \cdot \sqrt{\frac{2 \cdot Δ p}{ρ}}}{\sqrt{1 - {( \frac{{A}_{2}}{{A}_{1}} )}^{2}}}$$

For both configurations, the two pressure sensors 17 and 18a,b should preferably be positioned close to one another, because this can minimize any distortions in the result due to occurring pressure losses.

As an example, the first pressure sensor 18a,b and the second pressure sensor 17 are implemented as MEMS pressure sensors.

FIG. 4 schematically shows an illustration of a fluid channel through which fluid can flow. An equation for determining the fluid volume flow is derived below using the illustration of FIG. 4.

Based on Bernoulli's pressure equation for incompressible fluids. The equation is:

$${p}_{t} = p + \frac{ρ}{2} \cdot {v}^{2} = const .$$

$$\approx const .$$

Equalizing the (constant) total pressure at two points 1,2, it follows that:

$${p}_{1} + \frac{ρ}{2} \cdot {v}_{1}^{2} = {p}_{2} + \frac{ρ}{2} \cdot {v}_{2}^{2}$$

This results in the pressure difference Δp:

$$Δ p = {p}_{2} - {p}_{1} = \frac{ρ}{2} \cdot ( {v}_{1}^{2} - {v}_{2}^{2} )$$

The resulting constant mass flow is:

$$\dot{m} = ρ \cdot v \cdot A = const .$$

Solving for the flow velocity v, it follows that:

$$v = \frac{\dot{m}}{ρ \cdot A}$$

Substituting the flow velocity v in the equation for the pressure difference Δp results in:

$$Δ p = \frac{ρ}{2} \cdot {\dot{m}}^{2} \cdot ( {( \frac{1}{ρ \cdot {A}_{1}} )}^{2} - {( \frac{1}{ρ \cdot {A}_{2}} )}^{2} )$$

After a rearrangement, it follows that:

$$\dot{m} = \sqrt{\frac{\frac{2 \cdot Δ p}{ρ}}{( {( \frac{1}{ρ \cdot {A}_{1}} )}^{2} - {( \frac{1}{ρ \cdot {A}_{2}} )}^{2} )}}$$

After a rearrangement, it follows that:

$$\dot{m} = \frac{ρ \cdot {A}_{2}}{\sqrt{1 - {( \frac{{A}_{2}}{{A}_{1}} )}^{2}}} \cdot \sqrt{\frac{2 \cdot Δ p}{ρ}}$$

A volume flow Q can be determined as a quotient of mass flow to density:

$$Q = \frac{\dot{m}}{ρ}$$

After substitution, the equation for determining the fluid volume flow is as follows:

$$Q = \frac{{A}_{2}}{\sqrt{1 - {( \frac{{A}_{2}}{{A}_{1}} )}^{2}}} \cdot \sqrt{\frac{2 \cdot Δ p}{ρ}}$$

In the above derivation, Configuration A describes the limit case for v₁ tending to zero, which in the above equation A1 corresponds to tending to infinity.

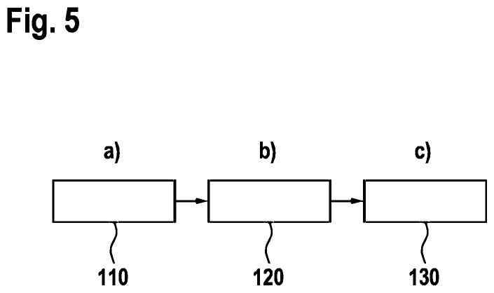

FIG. 5 schematically shows a sequence of a here presented method in a routine operating sequence. The method is used to determine at least a flow velocity or a fluid volume flow of a fluid flowing through an implanted vascular support system 10. The shown sequence of the method steps a), b) and c) with Blocks 110, 120 and 130 is only an example. Steps a) and b) in particular can also be carried out at least partially in parallel or even simultaneously. In Block 110, at least a static pressure or a total pressure in the region of the support system is determined by means of a first pressure sensor. In Block 120, at least a static pressure or a total pressure in the region of a fluid channel, which passes through the support system and through which fluid can flow, is determined by means of a second pressure sensor. In Block 130, at least the flow velocity or the fluid volume flow is determined using the pressures determined in Steps a) and b).

The solution presented here in particular enables one or more of the following advantages:

Continuous, accurate measurement of Qₚ using a system-integrated flow sensor. Qₚ is thus available as a control parameter of the support system, even outside the surgery scenario, with a quality comparable to that when using a CCO (Continuous Cardiac Output) thermodilution catheter.

When using pressure sensors, in particular MEMS pressure sensors, energy-efficient volume flow measurement is possible.

Compared to ultrasonic transducers, for example, a (MEMS) pressure sensor is very small.

What is claimed is:

1. A cardiac support system comprising:
a tubular structure configured to permit fluid to flow therethrough;
a first pressure sensor connected to the tubular structure and configured to determine at least one of a static pressure or a total pressure in a first region of the cardiac support system having a first cross-sectional area; and
a second pressure sensor connected to the tubular structure and configured to determine at least one of a static pressure or a total pressure in a second region of the cardiac support system having a second cross-sectional area of a different size relative to the first cross-sectional area.

2. The cardiac support system of claim 1, wherein the first pressure sensor is disposed in or on an outer side of the support system.

3. The cardiac support system of claim 1, wherein the first pressure sensor is disposed in or on an interior surface of the tubular structure.

4. The cardiac support system of claim 1, wherein the second pressure sensor is disposed in or on an interior surface of the tubular structure.

5. The cardiac support system of claim 1, wherein at least one of the first pressure sensor or the second pressure sensor is a MEMS pressure sensor.

6. The cardiac support system of claim 1, comprising an axial plane extending along a longitudinal axis of the cardiac support system, wherein the axial plane intersects the first pressure sensor and the second pressure sensor.

7. The cardiac support system of claim 1, wherein the first pressure sensor and the second pressure sensor are positioned along a common angular position around the cardiac support system.

8. The cardiac support system of claim 1, wherein the first region of the cardiac support system is positioned at a distal end of the tubular structure.

9. The cardiac support system of claim 1, wherein the first region is positioned distally of an inlet opening.

10. The cardiac support system of claim 9, wherein the inlet opening is disposed on a sidewall of the tubular structure.

11. The cardiac support system of claim 1, wherein the second region of the cardiac support system is positioned proximal to the first region.

12. The cardiac support system of claim 1 further comprising an inlet opening and an outlet opening axially displaced from the inlet opening, wherein the second region of the cardiac support system is positioned between the inlet opening and an outlet opening.

13. The cardiac support system of claim 12, wherein the inlet opening and the outlet opening are disposed on one or more side walls of the tubular structure.

14. The cardiac support system of claim 1, wherein the tubular structure comprises one or more side walls extending parallel to a longitudinal axis of the tubular structure.

15. The cardiac support system of claim 14, wherein the first pressure sensor and the second pressure sensor are disposed on the one or more side walls.
